(19) United States
(12) Patent Application Publication
(10) Pub. No.: US 2015/0165070 A1
(43) Pub. Date: Jun. 18, 2015

(54) MAGNETIC NANOPARTICLES DISPERSION, ITS PREPARATION AND DIAGNOSTIC AND THERAPEUTIC USE

(71) Applicants: CHARITÉ - UNIVERSITAETSMEDIZIN BERLIN, Berlin (DE); PHYSIKALISCH-TECHNISCHE BUNDESANSTALT (PTB), Braunschweig (DE)

(72) Inventors: Harald Kratz, Berlin (DE); Susanne Wagner, Mahlow (DE); Joerg Schnorr, Oranienburg (DE); Matthias Taupitz, Mahlow (DE); Monika Ebert, Mahlow (DE); Dietmar Eberbeck, Berlin (DE)

(21) Appl. No.: 14/390,585

(22) PCT Filed: Apr. 4, 2013
(86) PCT No.: PCT/EP2013/057144

§ 371 Date: Oct. 3, 2014

Related U.S. Application Data
(60) Provisional application No. 61/620,081, filed on Apr. 4, 2012.

(30) Foreign Application Priority Data
Apr. 4, 2012 (EP) 12163254.1

(51) Int. Cl. A61K 49/18; A61K 9/51; A61B 5/00; A61B 5/05; A61B 5/055

(52) U.S. Cl. CPC A61K 49/1851; A61B 5/0515; A61B 5/055; A61B 5/416; A61B 5/417; A61B 5/418; A61K 9/5161

(57) ABSTRACT

The present invention relates to magnetic particle dispersions comprising coated monocrystalline and/or polycrystalline single nanoparticles of iron oxides and nano-particulate aggregates (multi-core particles) thereof with improved nonlinear magnetization behavior and improved heating properties in alternating magnetic fields. When measured in a magnetic particle spectrometer (MPS) the particle dispersions show a pronounced overtone structure, especially in the higher harmonics, which surpasses all previously known particle systems many times over. Therefore, the dispersions are especially useful for applications such as MPI (magnetic particle imaging). In addition, the new particle dispersions are suitable for treatment of iron deficiency anemia and for applications in therapeutic hyperthermia, particularly passive partial-body hyperthermia or cell tracking and magnetic resonance imaging (MRI). Hence, the diagnostic and therapeutic use of the dispersions as well as pharmaceutical compositions of diagnostic or therapeutic interest comprising these dispersions are also objects of the present invention.

The present invention relates to magnetic particle dispersions comprising coated individual monocrystalline and/or polycrystalline nanoparticles of iron oxides and nanoparticulate aggregates (multi-core particles) thereof with improved nonlinear magnetization behavior and improved heating properties in alternating magnetic fields.

When measured in a magnetic particle spectrometer (MPS) the particle dispersions show a pronounced overtone structure, especially in the higher harmonics, which surpasses all previously known particle systems many times over. Therefore, the dispersions are especially useful for applications such as MPI (magnetic particle imaging). In addition, the new particle dispersions are suitable for treatment of iron deficiency anemia and for applications in therapeutic hyperthermia, particularly passive partial-body hyperthermia or cell tracking and magnetic resonance imaging (MRI). Hence, the diagnostic and therapeutic use of the dispersions as well as pharmaceutical compositions of diagnostic or therapeutic interest comprising these dispersions are also objects of the present invention.

In the field of technical applications the dispersions can be used for manufacturing electrets, pigments, functional coatings and for instance for final inspection in industrial production of non metal containing parts.

In the prior art a hugh number of different magnetic nanoparticles and aqueous dispersions or suspensions is comprising them is described. The described particles are so-called single-core particles or multi-core particles. For in vivo applications and for stabilization the magnetic particles are coated with a biocompatible shell, preferably with a biocompatible polymer.

The most widely used particles are particles based on magnetic iron oxids. Iron oxides based on magnetite (Fe₃O₄) and/or maghemite (γ-Fe₂O₃) exhibit ferrimagnetic behavior in magnetic fields. If nanoparticles of magnetite (Fe₃O₄) and/or maghemite (γ-Fe₂O₃) fall below a particular size, their behavior is superparamagnetic under certain circumstances, that is, they lack any residual magnetization (remanence) after turning off a previously activated magnetic field. Superparamagnetic iron oxide nanoparticles can be widely used e.g. in magnetic resonance tomography (MRT). The production and use of such particle preparations for use in MRT has been described in U.S. Pat. No. 5,424,419, DE 196 12 001 A1 and DE 4 428 851 A1, for example. But due to the fundamentally different physical phenomena which are used for imaging in the MRT and MPI methods, the suitability of a particle described as a contrast agent for MRT does not determine whether or not the particle is suitable for MPI.

Magnetic particle imaging (MPI) is a new imaging modality allowing direct representation and quantification of superparamagnetic iron oxide nanoparticles (SPIOs). The magnetization curve of SPIOs in magnetic fields is nonlinear, making it possible to measure overtones in addition to the incident fundamental frequency in alternating magnetic fields. These signals are specific to SPIOs and thus enable measurement with high sensitivity. Compared to MRT, the method provides potentially higher temporal and spatial resolution and therefore can be used not only in technical applications in the field of plastics, but also in non-invasive medical diagnostics, e.g. in diagnostic of cardiovascular diseases and particularly in the field of coronary heart diseases. Utilizing the potential of MPI requires special tracers exhibiting a particularly pronounced overtone structure in alternating magnetic fields, potentially resulting in high sensitivity of MPI measurements. A magnetic particle spectrometer (MPS) allows measurement of overtones generated by a sample in an alternating magnetic field.

Iron oxide nanoparticle preparations which are suitable for MPI are for instance described in EP 1 738 774 A1. These particles have a diameter of 20 nm to 1 μm with an overall particle diameter/core diameter ratio of less than 6. They are coated with a pharmaceutically acceptable polymer which is for instance carboxydextran or PEG. Carboxydextran stabilized iron oxid particles are the particles which are contained in the MRT contrast agent called Resovist®. From the examples of EP 1 738 774 A1 it is evident that Resovist® is also suitable for MPI.

The synthesis of Multicore nanoparticles is described in “Dutz, S, J H Clement, D Eberbeck, T Gelbrich, R Hergt, R Müller, J Wotschadlo, and Zeisberger. “Ferrofluids of Magnetic Multicore Nanoparticles for Biomedical Applications.” Journal of magnetism and magnetic materials 321, no. 10 (2009): doi:10.1016/j.jmmm.2009.02.073. It revealed that the dispersions which have been prepared according to the recipe given in this publication do not show a good stability.

Iron oxide nanoparticle preparations are also described for therapeutic hyperthermia. Therapeutic passive partial-body hyperthermia involves targeted incorporation of iron oxide-containing particle dispersions in tumors or tumor cells and heating by strong magnetic fields, thereby either directly damaging the tumor cells and/or increasing the effectiveness of administered chemotherapeutic agents. The use of particle dispersions containing iron oxides for therapeutic passive partial-body hyperthermia has been described for instance in WO 2006/125452. The strong magnetic fields being used not only cause heating of the particles, but also give rise to strong heating of metal-containing implants. Metallic dentures must therefore be removed from the patients prior to treatment.

Additionally, iron oxide nanoparticle preparations are described for treatment of iron deficiency anemia. Using oral iron substitution is not always sufficient for successful treatment of iron deficiency. In cases with strongly diminished serum iron levels a parenteral iron substitution drug is necessary to regulate the iron metabolism, normalize the iron stores and enhance the erythropoiesis.

On one hand iron is an absolutely essential element. On the other hand iron ions like ferric and ferrous iron are harmful on biological systems mainly due to their potential in inducing oxidative damage. Biomolecules like transferrin or ferritin are the main iron transport or storage form in mammalian biosystems. But parenteral iron drugs can overload this system, if the iron is released to fast. All clinically approved iron substitution drugs are based on iron carbohydrate compounds with iron in amorphous ferrihydrite, Akaganeite or as well magnetite or maghemite.

Some of the first parenteral iron substitution drugs are based on amorphous ferrihydrite dextran compounds like InFed®. Drawback of all these dextran based drugs is the carbohydrate sensitivity of dextran causing anaphylactic reactions to these drugs. Especially in patients with chronic kidney disease multiple iron substitutions end up in a senzitation during treatment courses.

Other drug with monomeric carbohydrate iron compounds like Ferrlecit® (ferric gluconate) or Venofer® (iron sucrose) are disadvantageous in releasing aggressive ferric and ferrous iron during blood circulation phase resulting in oxidative stress and cell damage mainly in the kidneys.

The drug Ferinject® based on a carboxypolymaltose with iron in the form of the ironoxy.hydroxide Akaganeite was thought to be free of any anaphylactic potential or iron ion toxicity. Disadvantageously, this drug induces a pathological and prolonged hypophosphatemia after intravenous administration.

The drug Feraheme® based on nanocrystallites of maghemite-magnetite with akaganeite coated with reduced carboxymethyldextran did not induce in rats dextran based anaphylactic reactions (WO 00/61191 A). Unfortunately in humans this adverse reaction still exists and was confirmed by an analytical antibody reaction test.

Currently all approved parenteral iron substitution drugs have one or more drawbacks leading to unwanted drug related adverse reactions in patients. Based on this knowledge the basic requirement for a parenteral iron substition drug could be summarized as follows:
no release of ferrous or ferric iron in serum during the blood circulation phase after administration
rapid uptake by cells involved in iron metabolism like spleen and liver macro phages
degradable by biological systems and release of iron as required
no induction of pathological hypophosphatemia

It was the object of the present invention to provide a stable magnetic particle dispersion with improved nonlinear magnetization behavior and improved heating properties in alternating magnetic fields in comparison to dispersions or suspensions described in the prior art. The dispersion of the present invention should be especially useful for both magnetic particle imaging (MPI) and therapeutic hyperthermia. In addition the dispersion of the present invention should also be useful for magnetic resonance imaging (MRI).

It was a further object of the present invention to provide a pharmaceutical composition for treatment of iron deficiency anemia, preferably by parenteral iron substitution, based on a magnetic particle dispersion which is stable over months and autoclavable.

The magnetic particle dispersion provided by the present invention comprises monocrystalline and/or polycrystalline single nanoparticles of iron oxides and at least 40 wt %, preferably at least 50 wt %, especially preferred 50-95 wt %, related to the total content of iron oxides of the dispersion, nanoparticulate aggregates (multi-core particles) thereof, wherein nanoparticles and nanoparticulate aggregates are coated with a pharmaceutically acceptable coating material selected from the group comprising a synthetic polymer, a carboxylic acid or hydroxycarboxylic acid, a monosaccharid, a disaccharid, a polysaccharid, or mixtures thereof. The dispersion shows nonlinear magnetization behaviour when subjected to an alternating magnetic field and at an incident fundamental frequency of 25 kHz and 10 mT flux density and 36.6° C. the value of the amplitude of the magnetic moment Aₖ generated by a dispersion having an iron content of 10 to 90 mmol Fe/l and measured with the magnetic particle spectrometer ranges at the third harmonic from 0.31045 to 15.79576 Am²/mol Fe, at the 21th harmonic from 3.78193·10⁻⁴ to 2.61583·10⁻² Am²/mol Fe and at the 51th harmonic from 3.98370·10⁻⁶ to 1.23649·10⁻⁴ Am²/mol Fe.

In a preferred embodiment of the invention the value of the amplitude of the magnetic moment Aₖ generated by a dispersion of the invention under the same conditions ranges at the 3rd harmonic from 0.31045 to 0.51994 Am²/mol Fe, at the 21th harmonic from 3.78193·10⁻⁴ to 7.76261·10⁻⁴ Am²/mol Fe and at the 51th harmonic from 3.9837·10⁻⁶ to 7.839487·10⁻⁶ Am²/mol Fe.

In another preferred embodiment of the invention the value of the amplitude of the magnetic moment Aₖ generated by a dispersion of the invention under the same conditions ranges at the 3^{rd} harmonic from 0.3104500 to 0.3631403 Am²/mol Fe, at the 21th harmonic from 3.78193·10⁻⁴ to 4.035128·10⁻⁴ Am²/mol Fe and at the 51th harmonic from 3.983704·10⁻⁶ to 7.839487·10⁻⁶ Am²/mol Fe.

For detection of the magnetic particle dispersion of the present invention by MPI, fields from 0.1 mT to 1 T and frequencies from 1 mHz to 1 MHz can be used.

In a preferred embodiment of the invention the magnetic particle dispersion comprises 50 to 95 wt % multicore particles related to the total content of iron oxides. Based on the multicore structure the particles do not have a notable magnetic moment in the absence of a magnetic field what diminishes the interaction of the particles and, hence, stabilizes the dispersion.

According to the present invention the magnetic particle dispersion preferably comprises 0-15 wt % of bivalent iron related to total iron content.

According to one embodiment of the present invention the pharmaceutically acceptable coating material can be a synthetic polymer or copolymer selected from the group consisting of polyethylenglycoles, polypropylenglycoles, polyoxyethylen and derivatives therefrom, polyoxypropylen and derivatives therefrom, polyamino acids, lactic and glycolic acid copolymers, or their mixtures.

According to another preferred embodiment of the present invention the pharmaceutically acceptable coating material is a polysaccharide selected from the group consisting of dextran, starch, chitosan, glycosaminoglycans (GAGs), starch phosphate, dextrin, maltodextrin, polymaltose, gum arabic, inulin, alginic acid and their derivatives, or mixtures thereof or a carboxylated polysaccharide, preferably a carboxymethylated polysaccharide. Dextran, dextrin, dextran derivatives or dextrin derivates are especially preferred. The dextran or dextrin derivates are selected from the group consisting of dextran or dextrin with carboxy groups, dextran or dextrin with aldehyde groups, biotinylated dextran or biotinylated dextrin, dextran or dextrin with SH-groups, reduced dextran, reduced carboxymethyldextran or mixtures thereof. Examples of GAGs which can be used as coating material according to the present invention include e.g. chondroitinsulfate, heparin, hyaluronan.

Carboxymethylated polysaccharides are also preferably used coating materials according to the present invention, especially carboxymethyldextrin and carboxymethyldextran (CMD).

In another preferred embodiment of the invention as pharmaceutically acceptable coating material a monosaccharide selected from the group consisting of D-mannitol, glucose, D-mannose, Fructose, Sorbitol, Inositol, their derivatives, or mixtures thereof is used, preferably D-mannitol.

According to the invention a combination of a polysaccharide as described above and a monosaccharid as described above may also be used as coating material for the nanoparticles, e.g. a combination of D-mannitol and carboxymethyldextran (CMD).

Carboxylic acids and hydroxycarboxylic acids selected from the group consisting of citric acid, malic acid, tartaric acid, gluconic acid, a fatty acid or mixtures thereof are also useful as coating materials according to the present invention. Preferably citric acid or D-gluconic acid can be used.

The nanoparticulate iron oxide crystals of the invention comprise magnetite (Fe₃O₄) and/or maghemite (γ-Fe₂O₃) and may additionally contain other iron oxides and iron mixed oxids with Mo, Cr, Mn, Co, Cu, Ni, Zn, or mixtures thereof. Preferably the iron oxide crystals of the dispersion of the present invention comprise magnetite and/or maghemite with an amount of at least 70 wt % related to the total content of iron oxide.

Stabilization of the nanoparticulate iron oxid crystals in water or organic solvents proceeds sterically and/or electrostatically as a result of the coating material surrounding the iron oxide crystals and the dispersion or suspension of the invention shows superparamagnetic properties in magnetic fields. In a preferred embodiment of the present invention the coated iron oxid crystals are dispersed or suspended in water, preferably they are dispersed in water.

The resulting particle dispersions of the invention comprisepolycrystalline and/or monocrystalline single nanoparticles having a size of from 2 to 50 nm as well as aggregates thereof embedded in a matrix of the coated material. The overall mean particle size of the single and multicore particles (hydrodynamic diameter) is between 10 and 80 nm. The individual polycrystalline and/or monocrystalline cores have sizes ranging up to the monodomain-multidomain limit, that means between 10 and 50 nm. The polycrystallites and multicore particles show the property of developing reduced anisotropy compared to monocrystalline nanoparticles of same size, resulting in an improvement of the energy transfer and/or MPS signal and in improved stability of the dispersions.

A further object of the present invention is the preparation method of the magnetic particle dispersions of the present invention and the magnetic particle dispersions obtainable by this method.

The preparation of the new particle dispersions comprises five steps a) to e) consisting of
a) alkaline precipitation of green rust (mixed ferrous/ferric hydroxide anion hydrates) from iron(II) salt solution with an alkaline solution, wherein the alkaline solution is added in an amount to ensure a pH value of the dispersion with the iron oxide nanoparticles obtained after step b) of 7.9 to 9.0,
b) oxidation with oxidants to form nanoparticulate iron oxid crystals comprising magnetite and maghemite
c) optionally, purification of the particles by magnetic separation
d) coating the particles with a pharmaceutically acceptable coating material and subsequent heating at 85° C. to 100° C. or autoclaving at 100 to 400° C. and at 1 to 240 bar to effect growth, aggregation and the size of the particles or
d′) heating the uncoated particles at 85° C. to 100° C. to effect growth, aggregation and the size of the particles and thereafter coating the particles with a pharmaceutically acceptable coating material and subsequent heating at 85° C. to 100° C. or autoclaving at 100 to 400° C. and at 1 to 240 bar to effect growth, aggregation and size of the particles and
e) fractionating the obtained particles by magnetic separation, washing them using ultrafiltration, dialysis, centrifugation and/or diafiltration until the filtrate or the supernatant has a conductivity value of less than 10 μS and re-fractionating them by magnetic separation without or after addition of alkali.

The synthesis in the alkaline range ensures that the nanoparticulate iron oxide crystals formed in step b) mainly consist of magnetite and maghemite, preferably to at least 70 wt %.

In a preferred embodiment of the invention step d) is performed, that means the particles are first coated and then heated. Here, besides the coated multi-core particles also coated single-core particles are present. It is preferred that the heating in step d) is performed for 2 to 36 hours, particularly for 4 to 20 hours, especially preferred for 7.5 to 15 hours, to ensure a good growth of the single-cores and aggregates. In case of step d′) it is sufficient to heat the uncoated particles for 30 minutes to 60 minutes. The heating after coating the particles with a pharmaceutically acceptable coating material is also performed as in step d) for 2 to 36 hours, particularly for 4 to 20 hours, especially preferred for 7.5 to 15 hours, to ensure a good growth of the single-cores and aggregates.

In a preferred embodiment of the invention the heating to effect aggregation and growth of the coated or uncoated particles according to step d) or d′) is carried out at 85 to 95° C., most preferred at about 90° C.

In a preferred embodiment of the invention an aqueous solution of FeCl₂ or of Fe(II) chloride tetrahydrate is used as iron(II) salt solution. Another Fe(II) salt which may be preferably used is FeSO₄ or Fe(II) sulphate heptahydrate. The alkaline solution for the precipitation in step a) is preferably a aqueous ammonium hydroxide or aqueous potassium hydroxide solution. Other bases which may be used are NaOH, Na₂CO₃, NaHCO₃, K₂CO₃, KHCO₃. The oxidation step b) is preferably carried out with a H₂O₂ solution, most preferred with a 5 wt % aqueous solution. Other oxidants which may be used are pure oxygen, atmospheric oxygen, NaNO₃, NaClO₄ and NaOCl.

In a preferred embodiment of the invention the coating in step d) or d′) is performed by adding the coating material at ambient temperature and stirring.

The described magnetic particle dispersions which are prepared according to the preparation method of the invention show a pronounced overtone structure surpassing known formulations in the higher harmonics when measured in a magnetic particle spectrometer. Therefore, the dispersions of the invention are potentially suitable for MPI (Magnetic Particle Imaging). As a result of the improved energy transfer, the new particle dispersions of the invention can also be used for applications in hyperthermic therapy of tumors. The new particle dispersions are easier to magnetize (more soft-magnetic) than those previously used. As a result, treatment can be performed using lower field strengths, thereby significantly reducing the side effects of the method. Improved transfer of energy from the external alternating magnetic fields to the iron oxide systems results in improved heating. Because of their good magnetic properties the described particle dispersions are also suitable for MRI applications. Additionally, it revealed that the aqueous particle dispersions of the invention are stable over more than 9 months until 12 months.

Therefore, the present invention also relates to a pharmaceutical composition comprising the magnetic particle dispersion of the invention and, optionally, pharmaceutically acceptable auxiliary substances. These auxiliary substances which can be added to diagnostic or therapeutic solutions are well known to the skilled expert. Such auxiliary substances are for instance preservatives, stabilizers, detergents, carriers, flavouring agents or phospholipides to encapsulate the magnetic particles in liposomes or micelles. They can be added to the dispersions of the invention without exception, if they are compatible with the dispersions. It is preferred that the pharmaceutical composition of the invention is a stabilized colloidal solution. In a special embodiment of the invention the pharmaceutical composition can contain surfactants like phospholipides or Pluronic® to incorporate the particles of the dispersion in micelles and liposomes. These magnetoliposomes and magnetomicelles have special characteristics and can be very useful for diagnosis and therapy. Therefore, a pharmaceutical composition which comprises magnetic particles encapsulated in liposomes or micelles is also an object of the present invention.

The pharmaceutical compositions of the present invention are especially useful in tumor staging and diagnosis of diseases e.g. of liver, spleen, bone marrow, lymph nodes, cardiovascular diseases, tumors and stroke by magnetic particle imaging (MPI) or magnetic resonance imaging (MRI). They are also useful for cell tracking or in hyperthermia, especially in passive partial-body hyperthermia and in tumor therapy by hyperthermia.

Additionally, the pharmaceutical compositions of the present invention comprising the magnetic particle dispersion as described are useful in treatment of iron deficiency anemia, preferably by parenteral iron substitution. This is possible, because the particles do not change during autoclaving what is the provision for providing a parenteral drug.

As it can be taken from the analytical and in vivo studies of the dispersion of the present invention all above mentioned requirements for parenteral iron substitution are fulfilled. It is assumed that the particulate character on the one hand and the close packed maghemite-magnetite crystals induce rapid phagocytosis without iron release. Additionally the multicore character of these type of iron oxide enables a large surface for intracellular iron metabolism enzymes which is not possible for large single core particles like e.g. in comparative example 2.

It could also be shown that antidextran antibodies do not cross-react with carboxymethyldextrine coated magnetic nanoparticles of the present invention what evidences the advantage of the carboxymethyldextrine coating. It also revealed that magnetic nanoparticle formulations of the invention are biodegradable in the liver of rats and show low phosphate binding capacity. The formulations of the invention show no side effects in rats at parenteral doses of 3 mmol Fe/kg body weight. The particles have good circulation half life in blood vessels of rats after intravenous injection what demonstrates that they may be suitable as contrast agents. It revealed that the multicore particles of the invention are better biodegradable than the single core particles (Compare Example 21). Additionally, the dispersions of the invention are autoclavable without loss or alteration of MPS signal what demonstrates the stability of the dispersion as a provision for drug formulation.

The invention also concerns a method for treating a patient in need of a tumor therapy comprising administering the magnetic particle dispersion or the pharmaceutical composition of the present invention directly to the diseased tissue of the patient and applying an alternating magnetic field (AMF) to the magnetic particle dispersion to inductively heat the magnetic particles. The magnetic particle dispersion can also be a component of an embolic agent or a mixture of embolic- and chemotherapeutic agent and administered by blood supply via a catheter.

The pharmaceutical composition of the present invention may be formulated for oral, parenteral, intratumoral, peritumoral, intralymphatic, in tissues, intravenous (IV), intra-arterial and intracerebral administration.

The invention also concerns a method for treating a patient with iron deficiency anemia in need of an iron substitution therapy comprising administering the magnetic particle dispersion of the invention or the pharmaceutical composition of the invention parenterally.

In the technical area the new particle dispersions of the invention can also be used for the manufacturing of electrets, pigments, functional coatings and for instance for final inspection in industrial production of non metal containing parts.

The following examples are offered to illustrate the preparation of the magnetic particle dispersions of the invention and their physical behaviour in an alternating magnetic field. The examples are not intended to be limiting in any respect.

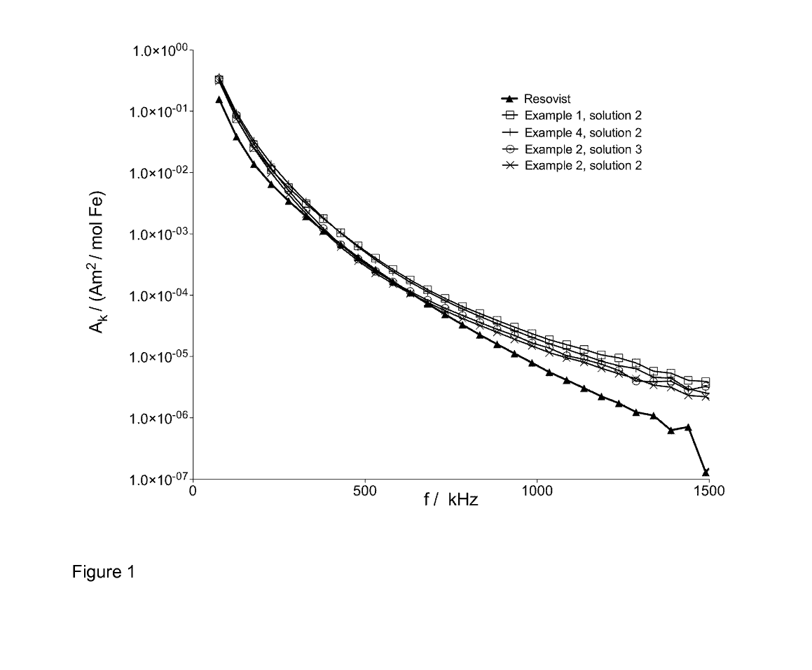

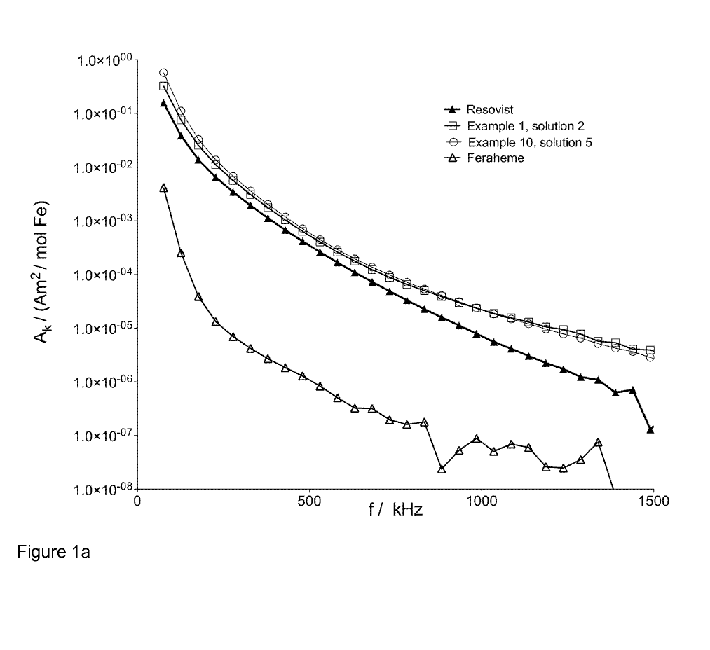

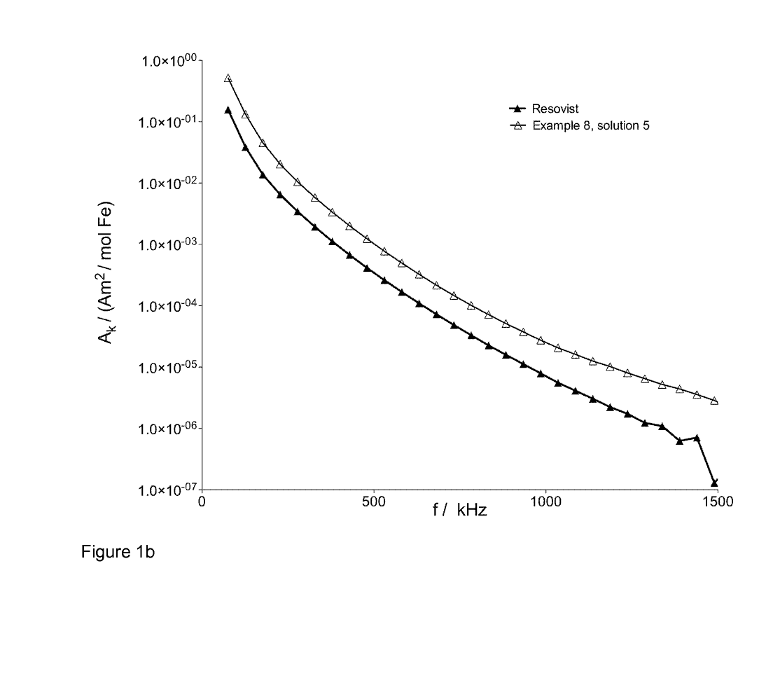

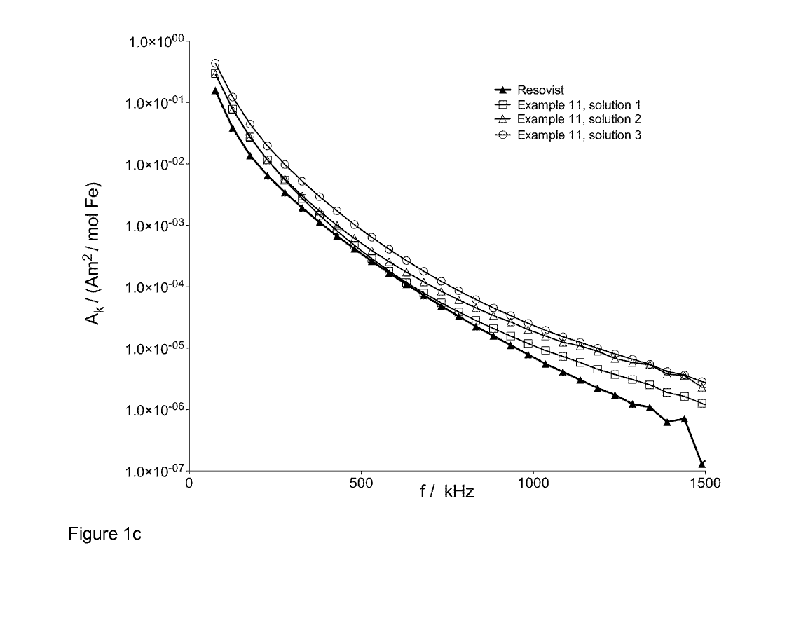

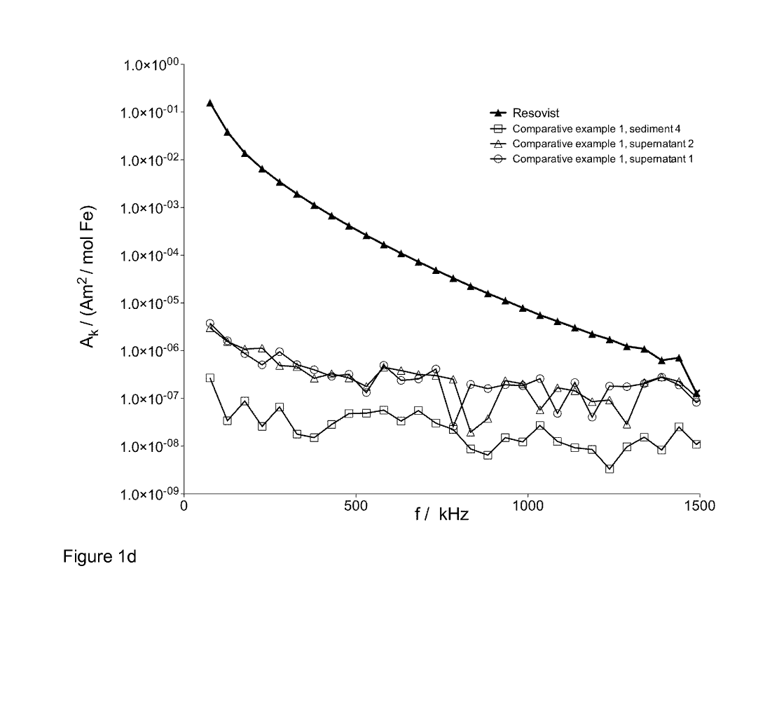

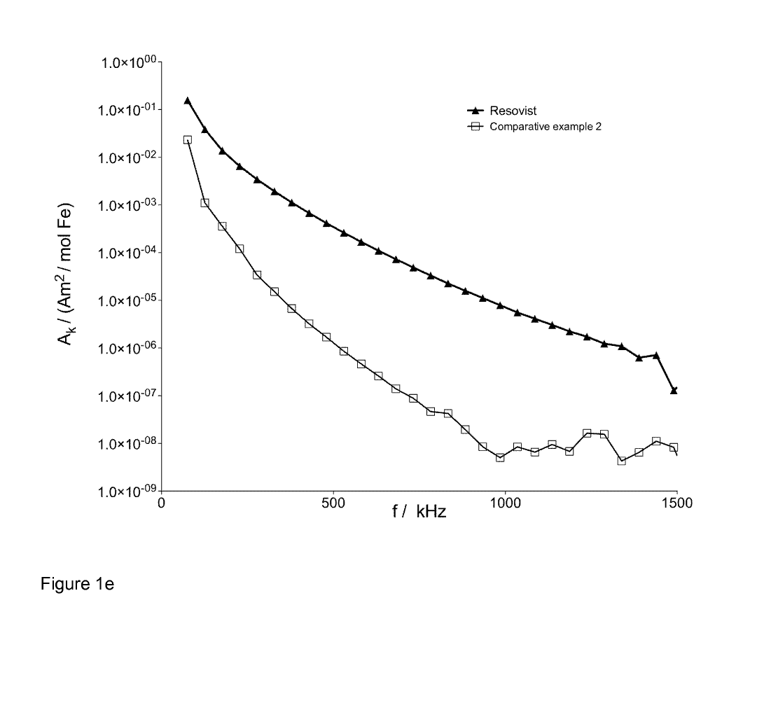

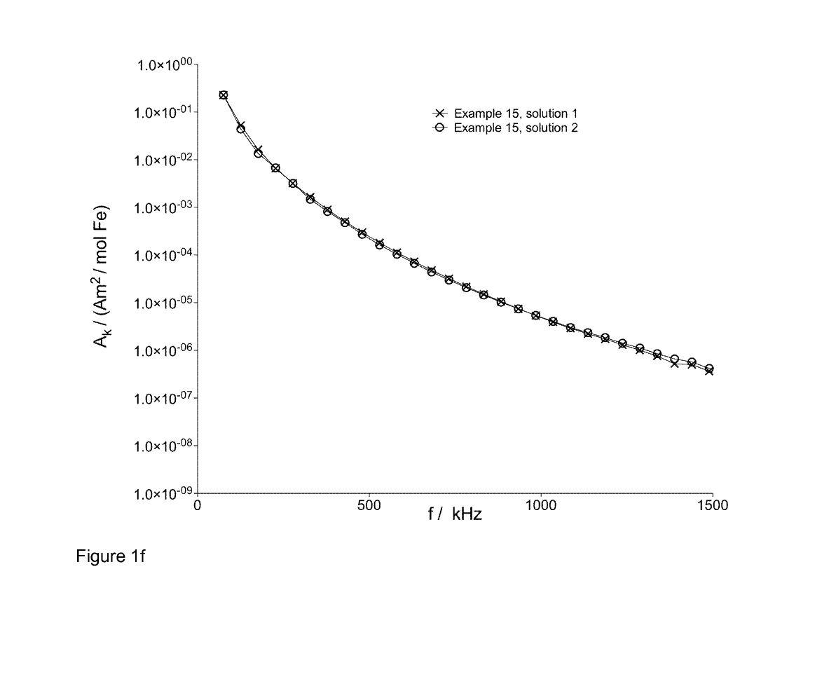

FIG. 1 shows MPS measurements (odd harmonics) of some of the dispersions according to the invention in comparison to Resovist®. FIG. 1a shows Example 1, solution 2, Example 10 solution 5, Resovist® and Feraheme®, FIG. 1b shows Example 8, solution 5 and Resovist®. FIG. 1c shows Example 11, solutions 1-3 and Resovist®. FIG. 1 d shows Comparative Example 1, sediment 4 and supernatants 1-2 and Resovist®. FIG. 1e shows Comparative Example 2 and Resovist®. FIG. 1a shows Example 15 solution 1 in comparison to Example 15, solution 2.

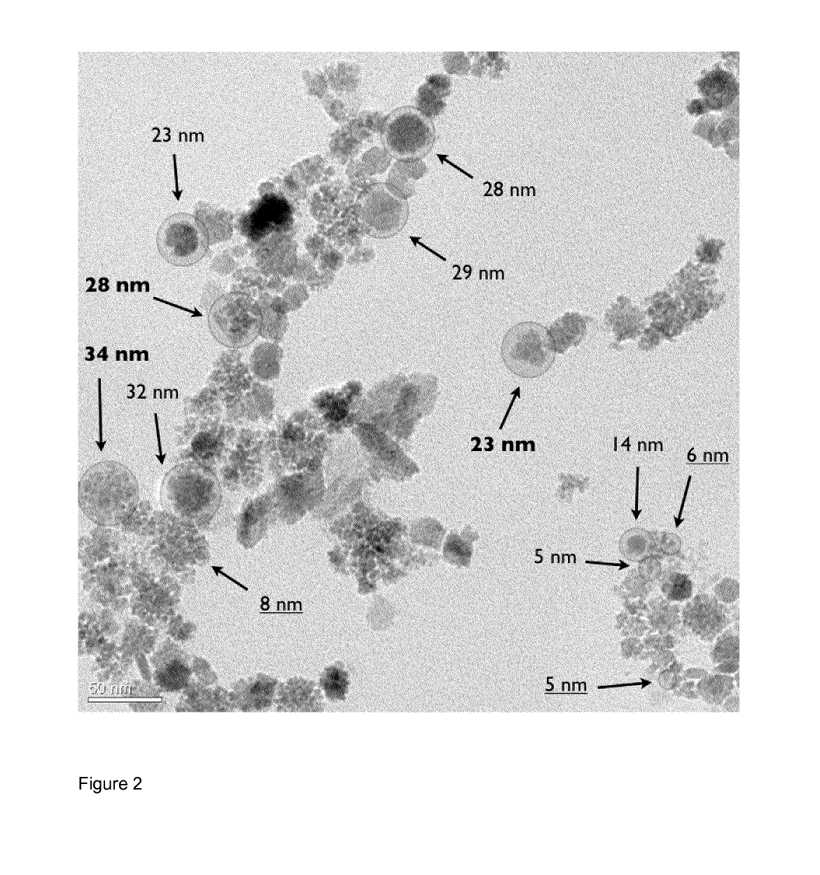

FIG. 2 shows the TEM image of solution 2 of Example 4.

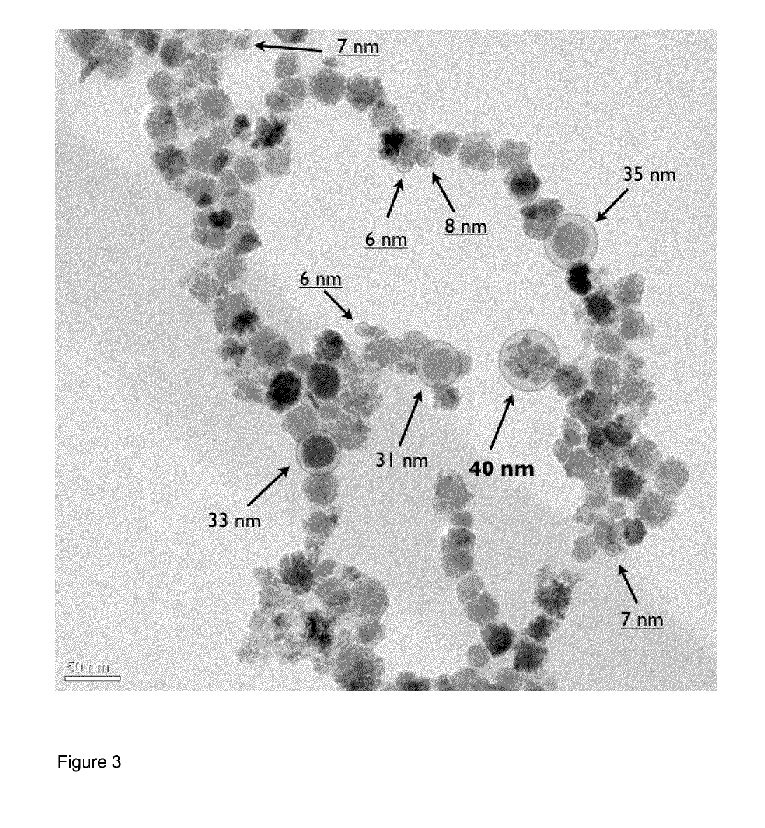

FIG. 3 shows the TEM image of solution 2 of Example 1.

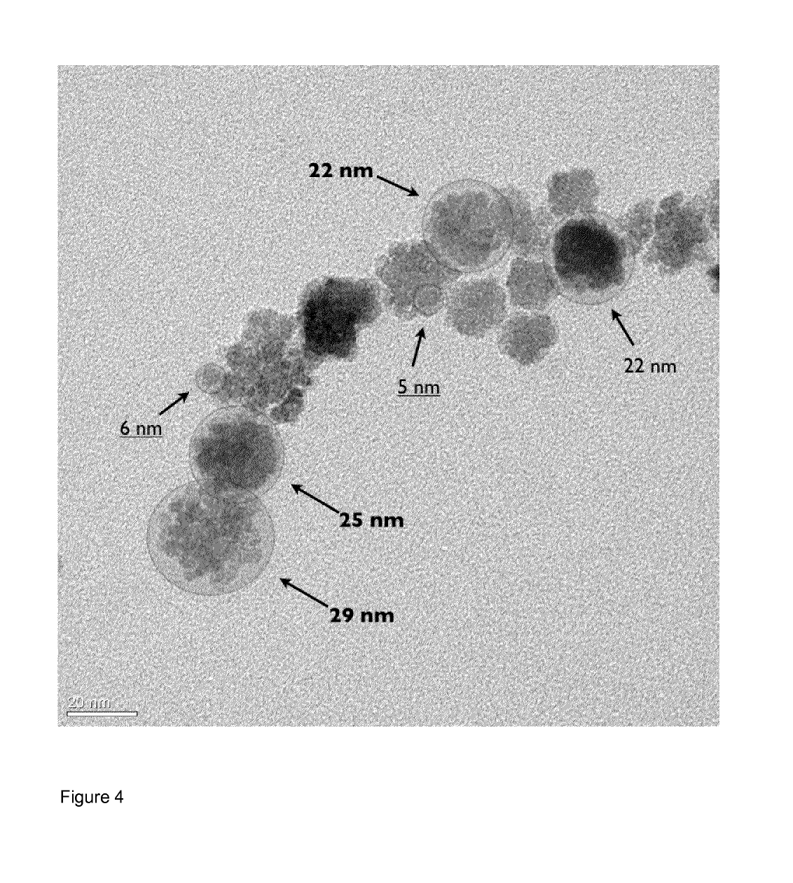

FIG. 4 shows the TEM image of solution 2 of Example 2.

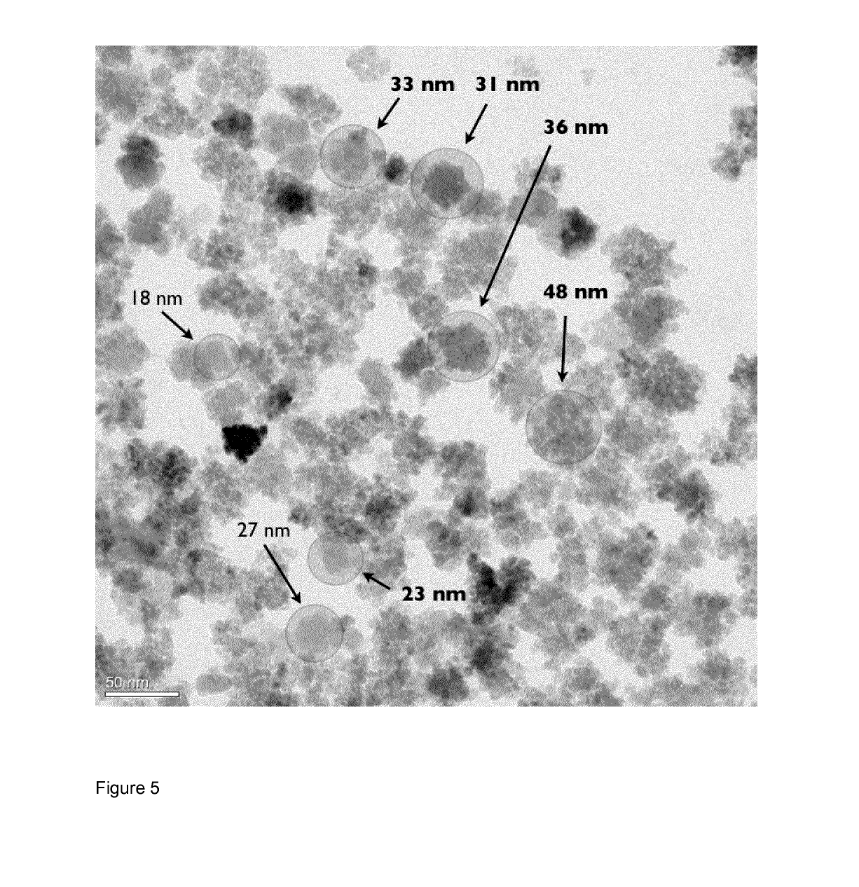

FIG. 5 shows the TEM image of solution 5 of Example 8.

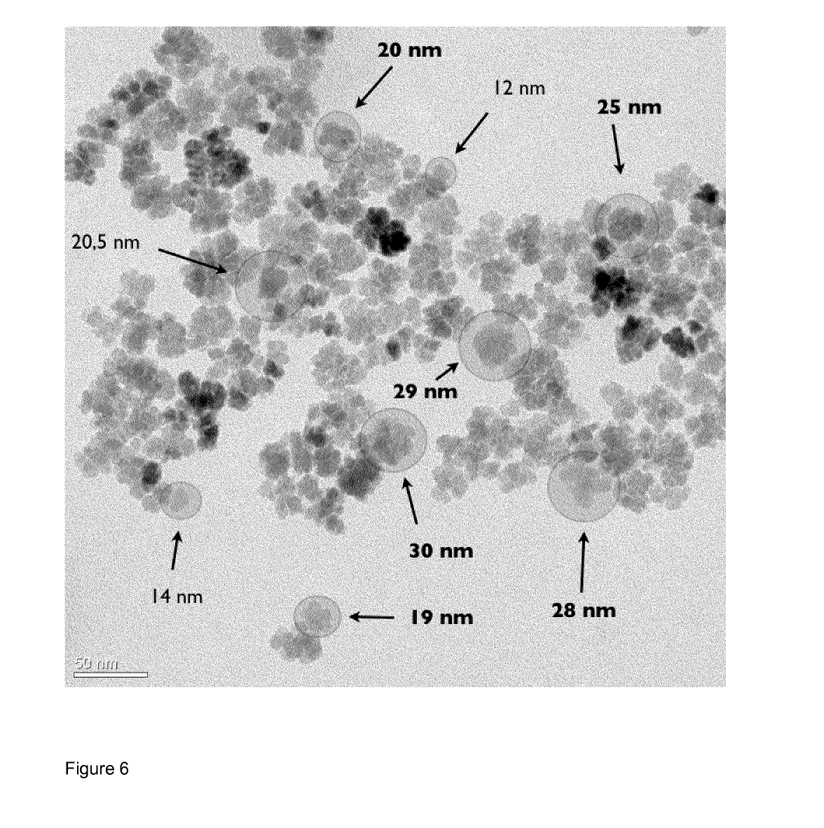

FIG. 6 shows the TEM image of solution 5 of Example 10.

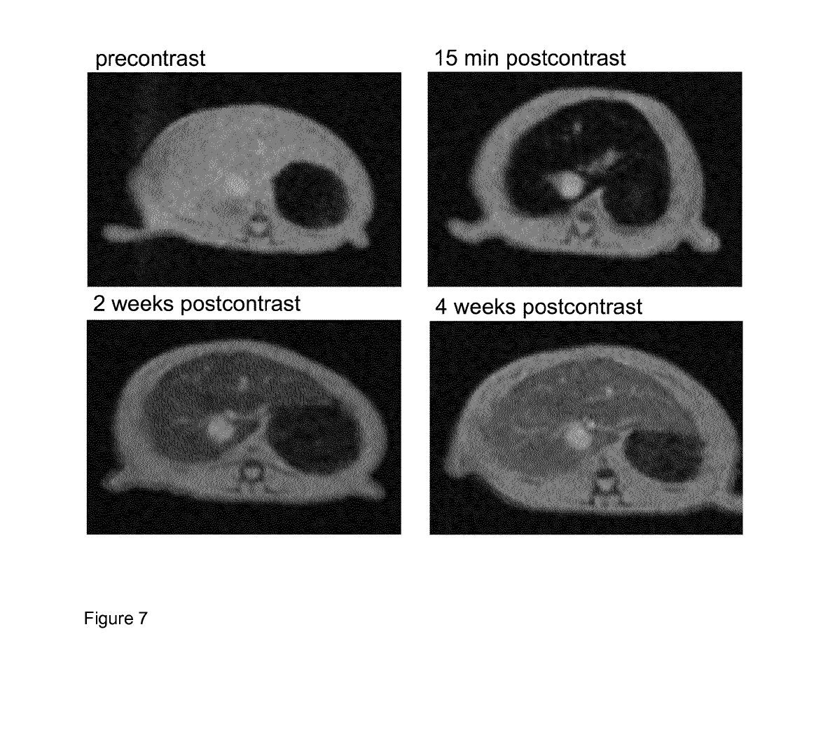

FIG. 7 shows magnetic resonance imaging of liver pharmacokinetic of example 14. Signal loss in liver showing rapid blood clearence and signal increase is showing degradation to non magnetic body iron store

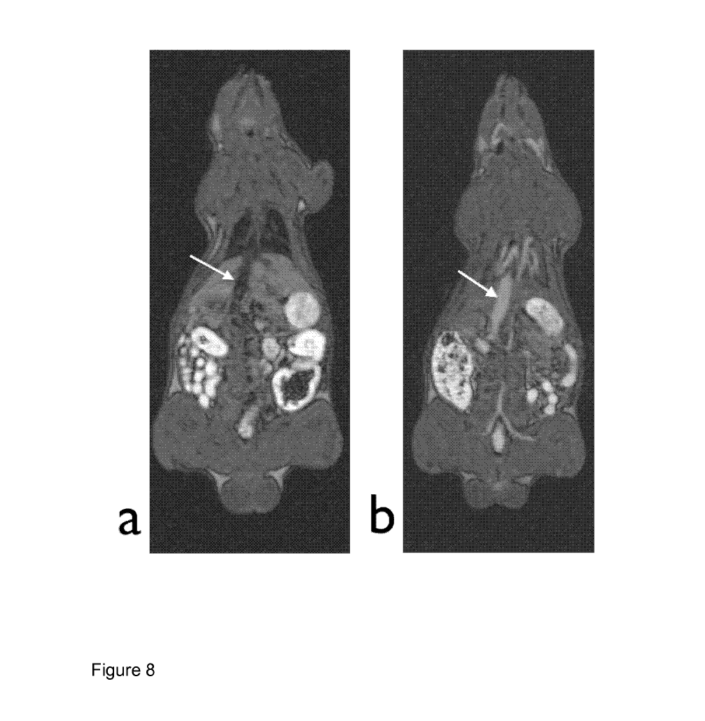

FIG. 8 shows T1 weighted (Tie) gradient echo (GRE) MR images of Male Sprague Dawley rats pre (a.) and post (b) injection of Example 13.

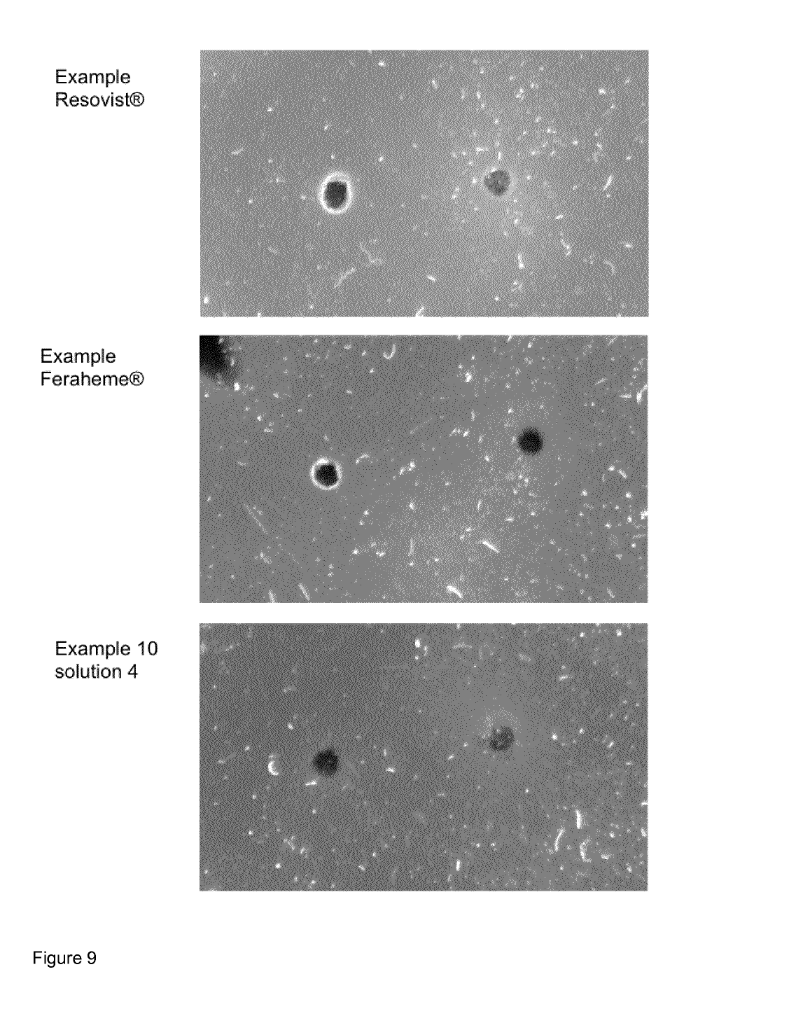

FIG. 9 shows results of Dextran-Antibody binding test of Example 10

In FIGS. 2 to 6 the sizes of the single core particles are given in normal writing, the multicore particles are marked in fat writing and the single cores of the multicore particles are depicted underlined.

### Preparation of the Magnetic Particle Dispersions

### EXAMPLE 1

### NH₄OH as Base, Polysaccharide Added

1.98 g of Fe(II) chloride tetrahydrate is dissolved in 200 ml of water at room temperature and under an air atmosphere (20% oxygen) over a period of 5 min with stirring. Thereafter, 2 ml of aqueous ammonium hydroxide (25 wt % NH₃) is added in one portion and stirred for about 5 min. 1 ml of aqueous hydrogen peroxide (5 wt %) is subsequently added in one portion and the solution is stirred for 10 min (pH value of the dispersion: 8.81). Thereafter, 4 g of carboxymethyldextran sodium salt (CMD-Na) is added and stirred for 10 min. The mixture is heated at 90° C. for 600 min. Subsequently, magnetic separation is performed for 20 min, the supernatant is decanted and the sediment suspended in 200 ml of water and subjected to another magnetic separation for 20 min, the supernatant is decanted, the sediment suspended in 200 ml of water, subjected to ultrasonic treatment for 5 min and magnetic separation for 20 min, and the supernatant is decanted. The sediment can be used for further workup. The supernatants are combined and washed with water via ultrafiltration using a Vivaflow 200 filter (100 kDa RC) until the filtrate has a conductivity value of less than 10 μS and subsequently concentrated to about 25 ml. The dispersion is placed on a magnet overnight and about 25 ml (solution 1) is pipetted off to obtain solution 1. The sediment is taken up with 25 ml of water and added dropwise with 0.85N aqueous potassium hydroxide solution until the pH of the solution is about 10. Following magnetic separation overnight, about 25 ml of solution (solution 2) is pipetted off to obtain solution 2. The sediment can be used for further workup.

analytical data of solution 1: iron content: 3.74 g Fe/l; content of bivalent iron in total iron: 11.47%; hydrodynamic size: 21.0-37.84 nm
analytical data of solution 2: iron content: 0.71 g Fe/l; content of bivalent iron in total iron: 12.61%; hydrodynamic size: 24.4-43.8 nm

### EXAMPLE 2

### KOH as Base, Aggregation Prior to Coating and Polysaccharide Addition

1.98 g of Fe(II) chloride tetrahydrate is dissolved in 200 ml of water at room temperature and under an air atmosphere (20% oxygen) over a period of 5 min with stirring. Thereafter, 22 ml of 0.85N aqueous potassium hydroxide solution is added in one portion and stirred for about 5 min. 1 ml of aqueous hydrogen peroxide (5 wt %) is subsequently added in one portion and the solution is stirred for 10 min (pH value of the dispersion: 8.03). Subsequently, magnetic separation is performed for 5 min, the supernatant is decanted and discarded. The sediment is taken up in 100 ml of water and placed on a magnet for another 10 min. After stirring for 10 min, the suspension is heated at 90° C. for 30 min and subsequently added with 4.2 g of carboxymethyldextran sodium salt (CMD-Na) and stirred for 5 min. The mixture is heated at 90° C. for 420 min. Subsequently, magnetic separation is performed for 20 min, the supernatant is decanted and the sediment suspended in 200 ml of water and subjected to another magnetic separation for 20 min, the supernatant is decanted, the sediment suspended in 200 ml of water, subjected to magnetic separation for 20 min, and the supernatant is decanted, the sediment is suspended in 200 ml of water and subjected to another magnetic separation for 20 min, the supernatant is decanted and the sediment can be used for further workup. The supernatants are combined and washed with water via ultrafiltration using a Vivaflow 200 filter (100 kDa RC) until the filtrate has a conductivity value of less than 10 μS and subsequently concentrated to about 30 ml. The dispersion is placed on a magnet overnight and about 20 ml (solution 1) is pipetted off to obtain solution 1. The sediment is taken up in 25 ml of water and added dropwise with 0.85N aqueous potassium hydroxide solution until the pH of the solution is about 10. Following magnetic separation overnight, about 23 ml of solution (solution 2) is pipetted off to obtain solution 2. The sediment is mixed with 25 ml of water and 280 mg of glycerophosphate and stirred for 5 min. Following magnetic separation overnight, about 30 ml of solution (solution 3) is pipetted off to obtain solution 3. The sediment can be used for further workup.

analytical data of solution 1: iron content: 2.20 g Fe/l; content of bivalent iron in total iron: 13.11%; hydrodynamic size: 18.2-32.7 nm
analytical data of solution 2: iron content: 1.12 g Fe/l; content of bivalent iron in total iron: 13.05%; hydrodynamic size: 18.2-32.7 nm
analytical data of solution 3: iron content: 0.48 g Fe/l; content of bivalent iron in total iron: 14.08%; hydrodynamic size: 24.0-37.8 nm

### EXAMPLE 3

### KOH as Base, Citric Acid Added

1.98 g of Fe(II) chloride tetrahydrate is dissolved in 200 ml of water at room temperature and under an air atmosphere (20% oxygen) over a period of 5 min with stirring. Thereafter, 22 ml of 0.85N aqueous potassium hydroxide solution is added in one portion and stirred for about 5 min. 1 ml of aqueous hydrogen peroxide (5 wt %) is subsequently added in one portion and the solution is stirred for 10 min (pH value of the dispersion: 8.05). Subsequently, magnetic separation is performed for 5 min, and the clear supernatant is decanted and discarded. The sediment is taken up in 50 ml of water and added with 1.1 g of citric acid monohydrate and stirred for 10 min at room temperature. The mixture is diluted to 90 ml with water and heated at 90° C. for 90 min. Subsequently, magnetic separation is performed for 10 min, the supernatant is decanted and the sediment suspended in 100 ml of water and subjected to another magnetic separation for 10 min, the supernatant is decanted, the sediment suspended in 100 ml of water, subjected to magnetic separation for 10 min, and the supernatant is decanted. The sediment can be used for further workup. The supernatants are combined and washed with water via ultrafiltration using a Vivaflow 200 filter (30 kDa PES) until the filtrate has a conductivity value of less than 10 μS and subsequently concentrated to about 30 ml. The dispersion is placed on a magnet overnight and about 20 ml (solution 1) is pipetted off to obtain solution 1. The sediment is taken up with 25 ml of water and added dropwise with 0.85N aqueous potassium hydroxide solution until the pH of the solution is about 11. Following magnetic separation overnight, about 20 ml of solution (solution 2) is pipetted off to obtain solution 2. The sediment can be used for further workup.

analytical data of solution 1: iron content: 0.78 g Fe/l; content of bivalent iron in total iron: 6.25%; hydrodynamic size: 7.5-15.7 nm
analytical data of solution 2: iron content: 0.56 g Fell; content of bivalent iron in total iron: 6.93%; hydrodynamic size: 11.7-21.0 nm

### EXAMPLE 4

### KOH as Base, Polysaccharide Added

3.96 g of Fe(II) chloride tetrahydrate is dissolved in 200 ml of water at room temperature and under an air atmosphere (20% oxygen) over a period of 5 min with stirring. Thereafter, 44 ml of 0.85N aqueous potassium hydroxide solution is added in one portion and stirred for about 10 min. 2 ml of aqueous hydrogen peroxide (5 wt %) is subsequently added in one portion and the solution is stirred for 10 min (pH value of the dispersion: 7.91). Subsequently, magnetic separation is performed for 5 min, and the supernatant is decanted and discarded. The sediment is taken up in 200 ml of water and placed on a magnet for another 15 min. Thereafter, 8 g of carboxymethyldextran sodium salt (CMD-Na) is added and stirred for 10 min at room temperature. The mixture is diluted with water to make a total volume of 250 ml and heated at 90° C. for 900 min. Subsequently magnetic separation with 100 ml of the resulting mixture is performed for 20 min, the supernatant is decanted and the sediment suspended in 200 ml of water and subjected to another magnetic separation for 20 min, the supernatant is decanted, the sediment suspended in 200 ml of water and subjected to magnetic separation for 20 min, the supernatant is decanted. The sediment is suspended in 200 ml of water and subjected to another magnetic separation for 20 min, the supernatant is decanted, the sediment is suspended in 200 ml of water and subjected to another magnetic separation for 20 min, the supernatant is decanted and the sediment is discarded or can be used for further workup. The supernatants are combined and washed with water via ultrafiltration using a Vivaflow 200 filter (100 kDa RC) until the filtrate has a conductivity value of less than 10 μS and subsequently concentrated to about 40 ml. The dispersion is placed on a magnet for 15 min, and about 35 ml is pipetted off (supernatant 1), the sediment (sediment 1) is preserved and supernatant 1 placed on a magnet overnight, and about 25 ml (solution 1) is pipetted off to obtain solution 1. The sediment 1 is taken up with 40 ml of water and added dropwise with 0.85N aqueous potassium hydroxide solution until the pH of the solution is about 10. Following magnetic separation for 15 min, about 42 ml of solution is pipetted off (supernatant 2), supernatant 2 is placed on a magnet overnight, and about 40 ml (solution 2) is pipetted off to obtain solution 2. The sediment can be used for further workup.

analytical data of solution 1: iron content: 2.03 g Fe/l; content of bivalent iron in total iron: 7.89%; hydrodynamic size: 18.2-28.2 nm
analytical data of solution 2: iron content: 1.05 g Fe/l; content of bivalent iron in total iron: 8.63%; hydrodynamic size: 18.2-32.7 nm

### EXAMPLE 5

### KOH as Base, Mono- and Polysaccharide Added

3.96 g of Fe(II) chloride tetrahydrate is dissolved in 200 ml of water at room temperature and under an air atmosphere (20% oxygen) over a period of 5 min with stirring. Thereafter, 44 ml of 0.85N aqueous potassium hydroxide solution is added in one portion and stirred for about 5 min. 2 ml of aqueous hydrogen peroxide (5 wt %) is subsequently added in one portion and the solution is stirred for 10 min (pH value of the dispersion: 8.42). Thereafter, 8 g of D-mannitol is added and stirred for 10 min at room temperature. The mixture is diluted with water to make a total volume of 250 ml and heated at 90° C. for 240 min. 150 ml of this mixture, while still hot, is placed on a magnet for 15 min and subsequently decanted. The sediment is taken up in 100 ml of water, 4.8 g of carboxymethyldextran sodium salt (CMD-Na) is added and the dispersion stirred for 10 min. The mixture is heated at 90° C. for 510 min. Subsequently, magnetic separation is performed for 20 min, the supernatant is decanted and the sediment suspended in 200 ml of water and subjected to another magnetic separation for 20 min, the supernatant is decanted, the sediment suspended in 200 ml of water and subjected to magnetic separation for 20 min, and the supernatant is decanted. The sediment can be used for further workup. The supernatants are combined and washed with water via ultrafiltration using a Vivaflow 200 filter (100 kDa RC) until the filtrate has a conductivity value of less than 10 μS and subsequently concentrated to about 40 ml. The sediment can be used for further workup.

analytical data:
iron content: 6.25 g Fell; content of bivalent iron in total iron: 2.29%

### EXAMPLE 6

### KOH as Base, Monosaccharide Added

1.98 g of Fe(II) chloride tetrahydrate is dissolved in 50 ml of water at room temperature and under an air atmosphere (20% oxygen) over a period of 5 min with stirring. Thereafter, 22 ml of 0.85N aqueous potassium hydroxide solution is added in one portion and stirred for about 5 min. 1 ml of aqueous hydrogen peroxide (5 wt %) is subsequently added in one portion and the solution is stirred for 10 min (pH value of the dispersion: 7.87). Subsequently, 4 g of D-gluconic acid sodium salt is added and stirred for 10 min at room temperature. The mixture is heated at 90° C. for 240 min. The mixture is added with 0.85N aqueous potassium hydroxide solution until the pH of the solution is about 10. Subsequently, magnetic separation is performed for 20 min, the supernatant is decanted and the sediment suspended in 100 ml of water and subjected to another magnetic separation for 20 min, the supernatant is decanted, the sediment suspended in 100 ml of water and subjected to magnetic separation for 20 min, and the supernatant is decanted. The sediment is suspended in 100 ml of water and subjected to another magnetic separation for 20 min, and the supernatant is decanted. The sediment can be used for further workup. The supernatants are combined and washed with water via ultrafiltration using a Vivaflow 200 filter (100 kDa RC) until the filtrate has a conductivity value of less than 10 μS and subsequently concentrated to about 40 ml. The sediment can be used for further workup.

analytical data:
iron content: 4.58 g Fell; content of bivalent iron in total iron: 1.02%

### EXAMPLE 7

### KOH as Base, Polysaccharide Added

3.96 g of Fe(II) chloride tetrahydrate is dissolved in 200 ml of water at room temperature and under an air atmosphere (20% oxygen) over a period of 5 min with stirring. Thereafter, 44 ml of 0.85N aqueous potassium hydroxide solution is added in one portion and stirred for about 10 min. 2 ml of aqueous hydrogen peroxide (5 wt %) is subsequently added in one portion and the solution is stirred for 10 min (pH value of the dispersion: 7.98). Subsequently, magnetic separation is performed for 12 min, and the supernatant is decanted and discarded. The sediment is taken up in 200 ml of water and placed on a magnet for another 15 min. Thereafter, 8 g of carboxymethyldextran sodium salt (CMD-Na) is added and stirred for 10 min at room temperature. The mixture is diluted with water to make a total volume of 250 ml and heated at 90° C. for 450 min. Thereafter subsequently, magnetic separation is performed for 23 min, the supernatant is decanted and the sediment suspended in 165 ml of water and subjected to another magnetic separation for 23 min, the supernatant is decanted, the sediment suspended in 165 ml of water and subjected to magnetic separation for 23 min, and the supernatant is decanted. The sediment is suspended in 165 ml of water and subjected to another magnetic separation for 23 min, the supernatant is decanted, and the sediment is discarded or can be used for further workup. The supernatants are combined and washed with water via ultrafiltration using a Vivaflow 200 filter (100 kDa RC) until the filtrate has a conductivity value of less than 10 μS and subsequently concentrated to about 67 ml.

The dispersion is placed on a magnet for 15 min, and about 60 ml is pipetted off (supernatant 1), the sediment (sediment 1) is preserved and supernatant 1 placed on a magnet overnight, and about 45 ml (solution 1) is pipetted off to obtain solution 1. The sediment 1 is taken up with 67 ml of water and added dropwise with 0.85N aqueous potassium hydroxide solution until the pH of the solution is about 10. Following magnetic separation for 15 min, about 80 ml of solution is pipetted off (supernatant 2), supernatant 2 is placed on a magnet overnight, and about 70 ml (solution 2) is pipetted off to obtain solution 2. The sediment can be used for further workup.

analytical data of solution 1: iron content: 5.86 g Fe/l; content of bivalent iron in total iron: 12.27%
analytical data of solution 2: iron content: 1.12 g Fe/l; solution 2: content of bivalent iron in total iron: 12.35%; hydrodynamic size: 21.04-43.82 nm

### EXAMPLE 8

### KOH as Base, Polysaccharide Added

3.96 g of Fe(II) chloride tetrahydrate is dissolved in 200 ml of water at room temperature and under an air atmosphere (20% oxygen) over a period of 5 min with stirring. Thereafter, 44 ml of 0.85 N aqueous potassium hydroxide solution is added in one portion and stirred for about 10 min. 2 ml of aqueous hydrogen peroxide (5 wt %) is subsequently added in one portion and the solution is stirred for 10 min. 1 ml of aqueous hydrogen peroxide (5 wt %) is subsequently added in one portion and the solution is stirred for 10 min (pH value of the dispersion: 8.05). Subsequently, magnetic separation is performed for 15 min, and the supernatant is decanted and discarded. The sediment is taken up in 100 ml of water. Thereafter, 8 g of carboxymethyldextran sodium salt (CMD-Na) is added and stirred for 10 min at room temperature. The mixture is diluted with water to make a total volume of 190 ml and heated at 90° C. for 480 min. Subsequently magnetic separation with the resulting mixture is performed for 20 min, the supernatant is decanted and the sediment suspended in 200 ml of water and subjected to another magnetic separation for 20 min, the supernatant is decanted, the sediment suspended in 200 ml of water and subjected to magnetic separation for 20 min, the supernatant is decanted. The sediment is suspended in 200 ml of water and subjected to another magnetic separation for 20 min, the supernatant is decanted, the sediment is discarded or can be used for further workup. The supernatants are combined and washed with water via ultrafiltration using a Vivaflow 200 filter (100 kDa RC) until the filtrate has a conductivity value of less than 10 mS and subsequently concentrated to about 40 ml. The dispersion is placed on a magnet overnight, and about 30 ml is pipetted off (solution 1), the sediment (sediment 1) is taken up with 25 ml of water and added dropwise with 0.85 N KOH solution until the pH of the solution is about 10. Following magnetic separation overnight, about 25 ml of solution is pipetted off (solution 2), the sediment (sediment 2) is taken up with 25 ml of water and placed on a magnet overnight, and 25 ml is pipetted off (solution 3), the sediment (sediment 3) is taken up with 25 ml of water and placed on a magnet overnight, and 25 ml is pipetted off (solution 4), the sediment (sediment 4) is taken up with 25 ml of water and placed on a magnet overnight, and 25 ml is pipetted off (solution 5), the sediment (sediment 5) can be used for further workup.

analytical data of solution 1: iron content: 8.71 g Fe/l; content of bivalent iron in total iron: 7.37%; hydrodynamic size: 15.7-28.2 nm
analytical data of solution 2: iron content: 8.66 g Fe/l; content of bivalent iron in total iron: 8.92%; hydrodynamic size: 21.0-37.8 nm
analytical data of solution 3: iron content: 2.40 g Fe/l; content of bivalent iron in total iron: 8.05%; hydrodynamic size: 21.0-37.8 nm
analytical data of solution 4: iron content: 1.56 g Fe/l; content of bivalent on in total iron: 9.32%; hydrodynamic size: 24.4-37.8 nm
analytical data of solution 5: iron content: 1.28 g Fe/l; content of bivalent iron in total iron: 8.79%; hydrodynamic size: 28.2-43.8 nm

### EXAMPLE 9

### Carboxymethyl Dextrin Sodium Salt

10.10 g sodium hydroxide was dissolved in 28 ml water. To this solution 8.35 g of Dextrin was added slowly and stirred for 10 min. Thereafter 140 ml isopropyl alcohol was added and the mixture stirred for 20 min at room temperature. After this 18.20 g of bromoacetic acid were added and the mixture was stirred rapidly at 70° C. for 120 min to solve the Dextrin completely and then stirred at room temperature overnight. The Solvent was removed in vacuo and the residue solved in 28 ml Water and the carboxymethyldextrin salt was precipitated with 252 ml of cold Methanol and the mixture was stored at 4° C. overnight. The mixture was subsequently filtrated, the precipitate washed with methanol and solved in 100 ml water, evaporated in vacuo, resolved in 50 ml water and dried at 60° C. Yield: 15.5 g Carboxymethyl dextrin sodium salt (CM-Dextrin-Na).

### EXAMPLE 10

### KOH as Base, Polysaccharide Added

3.96 g of Fe(II) chloride tetrahydrate is dissolved in 200 ml of water at room temperature and under an air atmosphere (20% oxygen) over a period of 5 min with stirring. Thereafter, 44 ml of 0.85 N aqueous potassium hydroxide solution is added in one portion and stirred for about 10 min. 2 ml of aqueous hydrogen peroxide (5 wt %) is subsequently added in one portion and the solution is stirred for 10 min. 1 ml of aqueous hydrogen peroxide (5 wt %) is subsequently added in one portion and the solution is stirred for 10 min (pH value of the dispersion: 8.36). Subsequently, magnetic separation is performed for 15 min, and the supernatant is decanted and discarded. The sediment is taken up in 100 ml of water. Thereafter, 8 g of carboxymethyldextrin sodium salt (CM-Dextrin-Na, Example 9) is added and stirred for 10 min at room temperature. The mixture is diluted with water to make a total volume of 190 ml and heated at 90° C. for 480 min. Subsequently magnetic separation with the resulting mixture is performed for 20 min, the supernatant is decanted and the sediment suspended in 200 ml of water and subjected to another magnetic separation for 20 min, the supernatant is decanted, the sediment suspended in 200 ml of water and subjected to magnetic separation for 20 min, the supernatant is decanted. The sediment is suspended in 200 ml of water and subjected to another magnetic separation for 20 min, the supernatant is decanted, the sediment is discarded or can be used for further workup. The supernatants are combined and washed with water via ultrafiltration using a Vivaflow 200 filter (100 kDa RC) until the filtrate has a conductivity value of less than 10 mS and subsequently concentrated to about 40 ml. The dispersion is placed on a magnet overnight, and about 30 ml is pipetted off (solution 1), the sediment (sediment 1) is taken up with 25 ml of water and added dropwise with 0.85 N KOH solution until the pH of the solution is about 10. Following magnetic separation overnight, about 25 ml of solution is pipetted off (solution 2), the sediment (sediment 2) is taken up with 25 ml of water and placed on a magnet overnight, and 25 ml is pipetted off (solution 3), the sediment (sediment 3) is taken up with 25 ml of water and placed on a magnet overnight, and 25 ml is pipetted off (solution 4), the sediment (sediment 4) is taken up with 25 ml of water and placed on a magnet overnight, and 25 ml is pipetted off (solution 5), the sediment (sediment 5) can be used for further workup.

analytical data of solution 4: iron content: 0.56 g Fe/l; content of bivalent iron in total iron: 5.60%; hydrodynamic size: 21.0-32.7 nm
analytical data of solution 5: iron content: 2.85 g Fe/l; content of bivalent iron in total iron: 4.65%; hydrodynamic size: 24.4-37.8 nm

### EXAMPLE 11

### KOH as Base, Polysaccharide Added

11.88 g of Fe(II) chloride tetrahydrate is dissolved in 600 ml of water at room temperature and under an air atmosphere (20% oxygen) over a period of 5 min with stirring. Thereafter, 132 ml of 0.85 N aqueous potassium hydroxide solution is added in one portion and stirred for about 10 min. 6 ml of aqueous hydrogen peroxide (5 wt %) is subsequently added in one portion and the solution is stirred for 10 min (pH value of the dispersion: 8.45). Subsequently, magnetic separation is performed for 15 min, and the supernatant is decanted and discarded. The sediment is taken up in 600 ml of water and placed on a magnet for another 15 min. Thereafter, 24.08 g of carboxymethyldextran sodium salt (CMD-Na) is added and stirred for 10 min at room temperature. The mixture is diluted with water to make a total volume of 750 ml and heated at 90° C. for 450 min. Thereafter subsequently, magnetic separation is performed for 23 min, the supernatant is decanted and the sediment suspended in 500 ml of water and subjected to another magnetic separation for 23 min, the supernatant is decanted, the sediment suspended in 500 ml of water and subjected to magnetic separation for 23 min, and the supernatant is decanted. The sediment is suspended in 500 ml of water and subjected to another magnetic separation for 23 min, the supernatant is decanted, and the sediment is suspended in 500 ml of water and subjected to another magnetic separation for 23 min, the supernatant is decanted, the sediment is discarded or can be used for further workup. The supernatants are combined and washed with water via ultrafiltration using a Vivaflow 200 filter (100 kDa RC) until the filtrate has a conductivity value of less than 10 μS and subsequently concentrated to about 200 ml.

The dispersion is placed on a magnet for 25 min, and about 180 ml is pipetted off (supernatant 1), the sediment (sediment 1) is preserved and supernatant 1 placed on a magnet overnight, and about 150 ml (solution 1) is pipetted off to obtain solution 1 and sediment 2. The sediment 1 is taken up with 180 ml of water and added dropwise with 0.85 N KOH solution until the pH of the solution is about 11.5. Following magnetic separation for 25 min, about 180 ml of solution is pipetted off (supernatant 2), supernatant 2 is placed on a magnet overnight, and about 155 ml (solution 2) is pipetted off to obtain solution 2 and sediment 3. The sediment 3 can be used for further workup. Sediment 2 is taken up with 180 ml of water and added dropwise with 0.85 N KOH solution until the pH of the solution is about 10.3. Following magnetic separation overnight, about 175 ml of solution is pipetted off (supernatant 3) to obtain solution 3 and sediment 4. The sediment 4 can be used for further workup. For Example 18 (phosphate adsorption and iron release in phosphate solution) Example 11 solution 2 was concentrated to 0.062 M Fe/L (solution 2 k) by centrifugation with 3112×g using Amicon Ultra-15 Centrifugal Filter Units (PLHK Ultracel-PL Membrane, 100 kDa).

analytical data of solution 1: iron content: 3.29 g Fe/l; content of bivalent iron in total iron: 3.48%; hydrodynamic size: 21.0-32.7 nm
analytical data of solution 2: iron content: 0.61 g Fe/l; content of bivalent iron in total iron: 2.43%; hydrodynamic size: 24.4-37.8 nm
analytical data of solution 3: iron content: 1.79 g Fe/l; content of bivalent iron in total iron: 2.01%; hydrodynamic size: 24.4-37.8 nm

### EXAMPLE 12

### Parenteral Formulation Version 1

Example 7, solution 2 was concentrated by centrifugation with 3112×g using Amicon Ultra-15 Centrifugal Filter Units (PLHK Ultracel-PL Membrane, 100 kDa). To 68 ml (0.171 M Fe/l) of the resulting solution 4.2 g D-Mannitol and 0.7 ml (2 g/l) aqueous sodium lactate was added. Thereafter the solution was passed through 0.2 μm (cellulose acetate) syringe filter (sterile filtration) and autoclaved at 120° C., 1 bar for 20 min. Iron content of the final solution: 0.165 M/l Fe

### EXAMPLE 13

### Parenteral Formulation Version 2

Example 7, solution 2 was concentrated by centrifugation with 3112×g using Amicon Ultra-15 Centrifugal Filter Units (PLHK Ultracel-PL Membrane, 100 kDa). To 7.5 ml (0.041 M Fe/l) of the resulting solution 0.48 g D-Mannitol and 80 μl (2 g/l) aqueous sodium lactate was added and water was added to bring the total volume to 8 ml. Thereafter the solution was passed through 0.2 μm (cellulose acetate) syringe filter (sterile filtration) and autoclaved at 120° C., 1 bar for 20 min. Iron content of the final solution: 0.039 M/l Fe

### EXAMPLE 14

### Parenteral Formulation Version 3

To 8 ml of Example 10, solution 5 (0.051 M Fe/l) 0.48 g D-Mannitol was added. Thereafter the solution was passed through 0.2 μm (cellulose acetate) syringe filter (sterile filtration) and autoclaved at 120° C., 1 bar for 20 min. Iron content of the final solution: 0.050 M/l Fe

### EXAMPLE 15

### Parenteral Formulation Version 4

Example 7, solution 2 was concentrated by centrifugation with 3112×g using Amicon Ultra-15 Centrifugal Filter Units (PLHK Ultracel-PL Membrane, 100 kDa). To 2 ml (0.170 M Fe/l) of the resulting solution (solution 1) 0.120 g D-Mannitol was added. Thereafter the solution was passed through 0.2 μm (cellulose acetate) syringe filter (sterile filtration) and autoclaved at 120° C., 1 bar for 20 min (solution 2). Iron content of the final solution (solution 2): 0.165 M/l Fe.

As it can be taken from FIG. 1f it revealed that solution 1 is autoclavable without loss or alteration of MPS signal what demonstrates the stability of the dispersion.

### EXAMPLE 16

### Dextran-Antibody Binding Test

Agarose test gel was prepared by mixing 8 ml 1% (wt/v) low gelling temperature agarose (Sigma-Aldrich, A9414) with 2 ml of the solution of the test iron drug compounds (example 10 solution 4, feraheme, positive dextran control, negative control) at a concentration of 40 μg Fe/ml in 0.9% sodium chloride solution. Agarose test compound mixture was filled in petri dishes. After gelling a 3 mm whole was prepared and the filled with 5 μl primary anti-dextran antibody Clone DX1 (StemCell Inc., Nr. 60026) in original concentration. After 2 day incubation at 4° C. the plate was washed three times with PBS buffer and the whole was filled with the secondary antibody Alexa Fluor 488 Goat Anti-mouse IgG1 (Life Technologies Inc., Nr. A21121). After 25 hour incubation plate was washed three times with PBS buffer solution an incubated further for three days in PBS buffer at 4° C.

Documentation was performed by image acquisition using Syngene G:Box (VWR company) with the image software Gene Snap Version 7.09.

Feraheme® and positive dextran control shows a precipitated ring, which did not occur in the plate with example 10 solution 4 and negative control (FIG. 9).

This Example shows that antidextran antibodies do not cross-react with the carboxymethyldextrine coated magnetic nanoparticles of Example 10 solution 4.

### EXAMPLE 17

### Pharmacokinetic of Example 13

Pharmacokinetic of example 13 was examined in three male Sprague Dawley Rats (Charles River, Germany) by magnetic resonance imaging at a Siemens Magnetom Syncro Maestro Class (Siemens, Germany) using a commercially available extremity coil. MR images of liver and spleen as the major target organs for iron metabolism were obtained with a 2D gradient echo sequence with a repetition time of 130 ms, an echo time of 5.4 ms and an flip angle of 40° with a slice thickness of 2 mm and an in plane resolution of 1×1 mm.

Rats were imaged before and subsequently 5 min, 15 min, 30 min, 60 min, 24 hours, 2 weeks and 4 weeks after intravenous administration of 0.045 mmol Fe/kg bw.

Signal intensities were measured in liver and background and SNR was calculated as follows: SNR=signal organ/signal background

Within 15 min SNR of liver declined from 12.34±1.5 to 0.86±0.2 and SNR of spleen declined from 10.97±3.2 to 1.6±4.1. SNR after 24 hours maintained at these low values. Within 14 days liver signal increased to an SNR of 6.01±2.6 and spleen SNR to 9.8±3.5. SNR of liver after 4 weeks was 8.21±2.0 and spleen SNR reached baseline values of 11.8±2.8.

In conclusion MRI reveals a rapid clearance by cells involved in iron metabolism and increase in signal shows good degradation of the iron crystal core magehemite structure in non-magnetic body iron compounds (FIG. 7).

This Example shows that the particle formulation of Example 13 is biodegradable.

### EXAMPLE 18

### Phosphate Adsorption and Iron Release in Phosphate Solution

Phosphate adsorption was determined in aqueous sodium phosphate solution at pH 7. A 40 mM phosphate solution (solution A) was prepared using sodium dihydrogen phosphate (S0751, Sigma-Aldrich, Munich, Germany). The pH of 7 was adjusted by adding either sodium hydroxide or hydrochloric acid.

Using 1 ml of solution A as aqueous medium, we prepared aqueous solutions of the production examples and comparative examples presented hereinafter, with an iron content of 0.1 mmol in 3 ml of total volume (solution B). Solution B was incubated for two hours at 37°. The solution was filtered with a 3 kDa Amicon ultracel Ultra-0.5 ml ultracentrifuge filter (9900×g). An aliquot of 0.5 ml of the diluted solution (1:500 with water) was mixed with 0.01 ml of 10% ascorbic acid, 0.02 ml of the molybdate reagent (25 ml 13% ammonium heptamolybdat+75 ml 9 M sulfuric acid+25 ml 0.35% potassium antimonyl tratrate trihydrate) and 0.47 ml bidestilled water. After 30 min of incubation at room temperature absorbance was measured at 880 nm (Specord 205, Analytic Jena AG, Germany). Phosphate was calculated based on a calibration curve (0-4 mg PO₄-/l). For iron analytic 0.2 ml of the filtrate was mixed with 0.1 ml 10% hydroxylamine hydrochloride and 0.7 ml 0.1% 1.10 phenanthroline (phenanthroline method). After 15 min incubation at room temperature absorbance was measured at 510 nm (Specord 205, Analytic Jena AG, Germany). Iron concentration was calculated based on a calibration curve (1-10 mg iron/ml).

TABLE 1

| Sample | PO₄-decrease in % | Iron release in % |
|---|---|---|
| Venofer ® | 5.0 | 0.0163 |
| Ferinject ® | 11.8 | 0.0030 |
| Example 10 solution 5 | 2.5 | 0.0030 |
| Comparative example 2 | 1.9 | 0.0030 |
| Example 11 solution 2 k | 4.0 | 0.0023 |

Table 1 shows the phosphate binding capacity of Examples 10 (solution 5), 11 (solution 2), 11 (solution 2 k) and Comparative Example 2 in comparison to Venofer® and Ferinject®. The data show that the phosphate binding capacity of dispersions of the invention is superior (lower value) to Venofer® and Ferinject®. The iron release of the dispersions of the invention is superior to Venofer® and comparable to Ferinject®

This Example shows the low phosphate binding capacity of Example 10 solution 5 and Example 11 solution 2k in comparison to Venofer®, Ferinject® and Comparative Example 2. A high phosphate binding capacity can cause hypophosphatemia.

### EXAMPLE 19

### Non Clinical Safety Testing

Tolerance was examined in male Sprague Dawley rats (Charles River, Germany) with a body weight of 300 g. Final drugs example 12 were administered at a doses of 3 mmol Fe/kg bw by slow bolus injection over a time period of two minutes intravenously into the lateral tail vein. Venofer® was tested at the same dosage. Before and at 5, 15, 30, 45, 60, 120, 180, 240 min and 24 h rats were set for one minute carefully in cleaned makrolon box to observe behavior and vital signs. Spontaneously released urine was collected and analyzed for pathological urine parameters using a Siemens Multstix® 8 SG. No signs of adverse reactions were observed. No change in urine parameters was found over the examined time period. It could be concluded, that 2 mmol Fe/kg is for the above tested examples the no observed adverse effect level (NOAEL). In contrary to this after administration of Venofer® rats showed no clinical signs of adverse effects but the urine Mulstix showed a dramatic increase in proteinuria above the level of 300 mg/I accompanied by a slight two plus hemoglobinuria. Pathological changes in urine parameters normalized completely 24 hours after administration.

This example shows the high tolerance of rats to Example 12 which showed no side effects at a parenteral dose of 3 mmol Fe/kg bw.

### EXAMPLE 20

### Short Term Blood Pharmacokinetic of Example 13

Short term blood pharmacokinetic examined by magnetic resonance imaging Male Sprague Dawley rats (300 g bw, Charles River Sulzfeld) were imaged before and every 5 minutes up to 30 minutes after intravenous administration of 0.045 mmol Fe/kg bw of example 13 at a Siemens Magnetom Syncro Maestro Class using a commercially available extremity coil in frontal section orientation with a T1 relaxation time weighted three dimensional gradient echo sequence (repetition time 5 ms, echo time 1.2 ms, flip anlfe 60°) with an inplane resolution of 0.6×0.6 mm and a slice thickness of 0.5 mm. Signal intensities in the caval vein was measured over the time course. Using a monoexponential fit according to a first order kinetic signal blood half life was calculated. After intravenous injection marked signal increase was observed only in blood vessel which remained with a half life of 4.4 minutes for example 13. This Example shows that the particle of example 13 have a circulation half life (in blood vessel) of 4.4 minutes after intravenous injection in rats.

### EXAMPLE 21

### Degradation Under Acidic Condition According to M. R, Jahn Et. Al. European Journal of Pharmaceutics and Biopharmaceutics 2011, 78, 480-491

Acidic hydrolysis of the iron compounds was examined in solutions of 0.9% sodium chloride/0.2375 M HCl with concentrations of 10 mg/L of the iron compounds. The mixtures were gently shaken for 50 h at room temperature, then filtered through a 3 kDa Amicon Ultra-0.5 ml ultracentrifuge filter at 5900×g and the iron content of the filtrate was measured by the phenanthroline method.

Table 2 shows rapid hydrolysis of examples 8 solution 5 and example 10 solution 5 in comparison to comparative example 2 under acidic conditions. It could be assumed, that the multicore shapes of example 8 solution 5 and example 10 solution 5 yields lager surface for hydrolysis reaction than larger crystals of comparative example 2. This could be a indication for a good biodegradability of the dispersions of the present invention.

TABLE 2

| Sample | Fe content mg/L |
|---|---|
| Example 10 solution 5 | 3.8 |
| Comparative example 2 | 2.4 |
| Example 8 solution 5 | 5.6 |

This Example shows rapid hydrolysis under acidic conditions of Example 10 solution 5 and Example 8 solution 5 in comparison to Comparative Example 2, which could be a indication for a good biodegradability.

### COMPARATIVE EXAMPLE 1

Multicore nanoparticles were prepared according to “Dutz, S, J H Clement, D Eberbeck, T Gelbrich, R Hergt, R Müller, J Wotschadlo, and Zeisberger. “Ferrofluids of Magnetic Multicore Nanoparticles for Biomedical Applications.” Journal of magnetism and magnetic materials 321, no. 10 (2009): doi:10.1016/j.jmmm.2009.02.073.

A solution of 1M NaHCO₃ was slowly added to a FeCl₂/FeCl₃ solution (total Feconcentration: 0.625 M; Fe²⁺/Fe³⁺ ratio=1/1.3) with a rate of 0.75 ml/min. When the pH value reached 8 addition of the bicarbonate solution was stopped. The resulting brownish precipitate was heated to 100° C. for 5 min under the release of CO₂. The prepared particles were washed with deionized water three times and the pH of the resulting suspension was adjusted to pH 2-3 by the addition of diluted HCl. Then the mixture was homogenized by ultrasonic treatment for a few seconds (Sonorex Digital 10P, Bandelin electronic) and then heated to 45° C. An aqueous solution of CMD (CMD sodium salt, Fluka) with an CMD/MCNP ratio of about 1:3 was added to the suspension and stirred for 60 min at 45° C. The coated particles were washed with de-ionized water and the resulting particle dispersion was centrifuged in a laboratory centrifuge (Labofuge 400R, Heraeus Sepatech) at 1029×g and 20° C. The sediment was stored and the supernatant was removed. The supernatant was centrifuged again with 1525×g. This procedure was repeated twice with 2521×g and 2958×g. Altogether, 8 fractions (4 sediments and 4 supernatants) were obtained.

It revealed that the magnetic properties of the obtained particle dispersions were not comparable with the one of the present invention (FIG. 1d). In addition the obtained particle dispersions showed no good stability combined with a high aggregation tendency, limiting the use as a parenteral drug.

### COMPARATIVE EXAMPLE 2

Multicore nanoparticles were prepared according to WO 03/0351 13 A1 (BERLIN HEART AG [DE]; GANSAU CHRISTIAN [DE]; BUSKE NORBERT [DE]; GOETZ) 1 May 2003 (2003-05-01), Page 15 Example 1 and Page 22 Example 17.

### Page 15 Example 1:

10 g of β-Cyclodextrin was mixed with 200 ml 2-Propanol and heated to 40° C. while stirring. Solutions of 1.) 6 g aqueous sodium hydroxide in 20 ml water and 2.) 15 g chloroacetic acid sodium salt in 40 ml water were added and the resulting solution was heated to 70° C. and stirred rapidly for 90 minutes. After cooling to room temperature the 2-Propanol phase (upper phase) was decanted, the residue (the lower phase) was adjusted to a pH of 8 and treated with 120 ml of Methanol to precipitate the raw product. The methanolic solution was decanted, the precipitate solved in 100 ml of water and passed through an acidic ion exchange resin (Dowex 50). The resulting solution was dialysed overnight and lyophilised to get Carboxymethyl-3-cyclodextrin.

### Page 22 Example 17:

20 g of Fe(II) chloride was dissolved in 300 ml water, heated to 70° C. and treated with 40 ml 6M aqueous potassium hydroxide solution while stirring. Thereafter 9.7 ml of a 10 wt % aqueous solution of aqueous hydrogen peroxide was slowly added and the resulting solution was stirred for 40 min at 70-75° C. The Precipitate was separated by a magnet, washed with water several times, mixed with 200 ml water. The pH of the mixture was adjusted to a pH of 1.5 and the mixture was heated to 50° C. Then a solution of 1.5 g Carboxymethyl-β-cyclodextrin (Page 15 Example 1) and 40 ml water was added and the resulting mixture was stirred at 50° C. for 30 minutes. The suspension was separated by a magnet, washed with water several times, suspended in 40 ml water, neutralised with 3M aqueous sodium hydroxide solution and dispersed with ultrasound.

analytical data: iron content: 34.85 g Fe/1; content of bivalent iron in total iron: 15.57%; hydrodynamic size: 78.8-141.8 nm

It revealed that the magnetic properties of the obtained particle dispersions were not comparable with the one of the present invention (FIG. 1 e). In addition the obtained particle dispersions showed no good stability combined with a high aggregation tendency, limiting the use as a parenteral drug.

### MPS Measurements of the Dispersions of the Invention in Comparison to Resovist®

The undiluted samples of Examples 1 (solution 2), 2 (solutions 2 and 3) 4 (solution 2), 7 (solution 2), 8 (solution 5) and 10 (solution 5) were measured in a magnetic particle spectrometer (MPS) (Bruker Biospin, Germany) at 10 mT, 25.2525 kHz, 36.6° C. and for 10 s. For comparison the commercially available Resovist® dispersion was diluted with water to give 33 mmol Fe/L and measured under the same conditions. The measurements were carried out in PCR tubes of Life Technologies with a volume of the samples of 30 μl. For evaluation the obtained measured value of each harmonic which corresponds to the amplitude of the magnetic moment was normalized to the respective iron content of each sample and given as Aₖ in Am²/mol Fe. The results are depicted in Table 3 and FIG. 1. Only odd harmonics are shown.

TABLE 3

| | | 3rd harmonic | 21th harmonic | 51th harmonic |
|---|---|---|---|---|
| Sample | Iron content | (75.75 kHz) | (530.30 kHz) | (1287.88 kHz) |
| Example 1, | 0.013M Fe/L | 0.3269231 | 0.0004035128 | 7.839487*10⁻⁶ |
| solution 2 | | | | |
| Example 4, | 0.019M Fe/L | 0.3631403 | 0.000378193 | 6.347895*10⁻⁶ |
| solution 2 | | | | |
| Example 2, | 0.090M Fe/L | 0.3389185 | 0.0002457926 | 3.983704*10⁻⁶ |
| solution 3 | | | | |
| Example 2, | 0.020M Fe/L | 0.3104500 | 0.0002292000 | 4.353333*10⁻⁶ |
| solution 2 | | | | |
| Example 7, | 0.020M Fe/L | 0.2997000 | 0.000331300 | 4.921333*10⁻⁶ |
| solution 2 | | | | |
| Example 8, | 0.023M Fe/L | 0.5199420 | 0.0007762609 | 6.468551*10⁻⁶ |
| solution 5 | | | | |
| Example 10, | 0.051M Fe/L | 0.5747843 | 0.0004469412 | 6.511046*10⁻⁶ |
| solution 5 | | | | |
| Resovist ® | 0.033M Fe/L | 0.1579576 | 0.0002615828 | 1.236485*10⁻⁶ |

As it can be taken from Table 3 the dispersions of the invention are superior to the Resovist® preparation. E.g. solution 2 of Example 1 is superior to the Resovist® preparation by a factor of two at the 3rd harmonic and by a factor of 6 at the 51th harmonic.

1. A magnetic particle dispersion comprising monocrystalline and/or polycrystalline single nanoparticles of iron oxides and at least 40 wt %, related to the total iron content of the dispersion, nanoparticulate aggregates thereof, wherein nanoparticles and nanoparticulate aggregates are coated with a pharmaceutically acceptable coating material selected from the group comprising a polysaccharide, a carboxylic or hydroxycarboxylic acid selected from the group consisting of citric acid, malic acid, tartaric acid, gluconic acid, a fatty acid or mixtures thereof, a monosaccharide, a disaccharide, a or mixtures thereof, the dispersion showing nonlinear magnetization behaviour when subjected to an alternating magnetic field and at an incident fundamental frequency of 25.25 kHz, 10 mT flux density and 36.6° C. the value of the amplitude of the magnetic moment Aₖ generated by a dispersion having an iron content of 10 to 90 mmol Fe/1 and measured with the magnetic particle spectrometer ranges at the third harmonic from 0.31045 to 15.79576 Am²/mol Fe, at the 21th harmonic from 3.78193·10⁻⁴ to 2.61583·10⁻² Am²/mol Fe and at the 51th harmonic from 3.9837·10⁻⁶ to 1.23649·10⁻⁴ Am²/mol Fe.

2. The magnetic particle dispersion according to claim 1, wherein the mean particle size (hydrodynamic diameter) of the single nanoparticles and nanoparticulate aggregates is between 10 and 80 nm.

3. The magnetic particle dispersion according to claim 1, wherein the pharmaceutically acceptable coating material is a polysaccharide selected from the group comprising dextran, starch, starch phosphate, chitosan, glycosaminoglycans, dextrin, maltodextrin, polymaltose, gum arabic, inulin, alginic acid and their derivatives, or mixtures thereof.

4. The magnetic particle dispersion according to claim 1, wherein the pharmaceutically acceptable coating material is a carboxylated polysaccharide.

5. The magnetic particle dispersion according to claim 1, wherein the pharmaceutically acceptable coating material is a monosaccharide selected from the group consisting of D-mannitol, glucose, D-mannose, Fructose, Sorbitol, and Inositol.

6. The magnetic particle dispersion according to claim 1, wherein the pharmaceutically acceptable coating material is a hydroxycarboxylic acid selected from citric acid or D-gluconic acid.

7. The magnetic particle dispersion according to claim 1, wherein the nanoparticulate iron oxides comprise magnetite (Fe₃O₄), maghemite (γ-Fe₂O₃), iron mixed oxids with Mo, Cr, Mn, Co, Cu, Ni, Zn, or mixtures thereof.

8. A method for preparing a magnetic particle dispersion according to claim 1, wherein the method comprises five steps a) to e) consisting of
a) alkaline precipitation of green rust from iron(II) salt solution with an alkaline solution, wherein the alkaline solution is added in an amount to ensure a pH value of the dispersion with the iron oxide nanoparticles obtained after step b) of 7.9 to 9.0,
b) oxidation with oxidants to form nanoparticulate iron oxid crystals comprising magnetite and maghemite
c) optionally, purification of the particles by magnetic separation
d) coating the particles with a pharmaceutically acceptable coating material and subsequent heating at 85 to 100° C. or autoclaving at 100 to 400° C. and at 1 to 240 bar to effect growth, aggregation and the size of the particles
or
d) heating the uncoated particles at 85 to 100° C. to effect growth, aggregation and the size of the particles and thereafter coating the particles with a pharmaceutically acceptable coating material and subsequent heating at 85 to 100° C. or autoclaving at 100 to 400° C. and at 1 to 240 bar to effect growth, aggregation and the size of the particles
and
e) fractionating the obtained particles by magnetic separation, washing them using ultrafiltration, dialysis, centrifugation and/or diafiltration until the filtrate or the supernatant has a conductivity value of less than 10 μS and re-fractionating them by magnetic separation without or after addition of alkali.

9. The method according to claim 8, wherein an aqueous solution of Fe(II) chloride tetrahydrate or Fe(II) sulfate hepathydrate is used as iron(II) salt solution.

10. The method according to claim 8, wherein the oxidation step b) is performed with H₂O₂, pure oxygen, atmospheric oxygen, NaNO₃, NaClO₄ or NaOCl as oxidant.

11. The method according to claim 8, wherein the heating to effect size, aggregation and growth of the coated and uncoated particles in step d) or d′) is carried out at 85 to 95° C.

12. The method according to claim 7, wherein the heating in step d) is performed for 2 to 36 hours.

13. A pharmaceutical composition comprising a magnetic particle dispersion of claim 1 and pharmaceutically acceptable auxiliary substances.

14. The magnetic particle dispersion of claim 1 for use in diagnosis of diseases and tumor staging by magnetic particle imaging (MPI) or magnetic resonance imaging (MRI) or for cell tracking by MPI.

15. (canceled)

16. (canceled)

17. The magnetic particle dispersion of claim 1 for use in treatment of iron deficiency anemia.

18. The magnetic particle dispersion according to claim 4, wherein the pharmaceutically acceptable coating material is a carboxymethylated polysaccharide.

19. The magnetic particle dispersion according to claim 4, wherein the pharmaceutically acceptable coating material is carboxymethyldextran or carboxymethyldextrin.

20. The magnetic particle dispersion according to claim 7, wherein the nanoparticulate iron oxides comprise magnetite and/or maghemite.

21. The magnetic particle dispersion according to claim 7, wherein the nanoparticulate iron oxides comprise magnetite and/or maghemite with an amount of at least 70 wt %, related to the total content of iron oxide.

22. The magnetic particle dispersion of claim 14, for use in diagnosis of spleen diseases, bone marrow diseases, lymph node diseases, cardiovascular diseases, tumors and stroke.
